(19) United States
(12) Patent Application Publication
(10) Pub. No.: US 2004/0044078 A1
(43) Pub. Date: Mar. 4, 2004

(54) Cosmetic and/or pharmaceutical emulsions

(76) Inventors: Rolf Kawa, Monheim (DE); Achim Ansmann, Erkrath (DE)

Correspondence Address: COGNIS CORPORATION, PATENT DEPARTMENT, 300 BROOKSIDE AVENUE, AMBLER, PA, 19002, US

(21) Appl. No.: 10/433,106

(22) PCT Filed: Nov. 20, 2001
(86) PCT No.: PCT/EP01/13387

(30) Foreign Application Priority Data
Nov. 29, 2000 (DE) 10059239.2

(51) Int. Cl. A61K 7/075; A61K 7/08; A61K 31/20

(52) U.S. Cl. 514/558; 424/70.22

(57) ABSTRACT

A cosmetic and/or pharmaceutical composition containing: (a) an oil component; (b) a viscosity modifier selected from the group consisting of a C₆₋₂₂ hydroxyfatty acid, a salt of a C₆₋₂₂ hydroxyfatty acid, and mixtures thereof; and (c) water.

### FIELD OF THE INVENTION

This invention relates generally to cosmetic products and, more particularly, to cosmetic and/or pharmaceutical emulsions containing long-chain hydroxyfatty acids and/or salts thereof and to their use in waterproof sun protection preparations and as viscosity adjusters.

### PRIOR ART

An important factor in the modern formulation development of cosmetic and pharmaceutical emulsions is inter alia the choice of the emulsifiers. Not only should the emulsifiers used be capable of stabilizing the phases and the viscosity of emulsions over a prolonged period, even when used in low concentrations, they are also expected favorably to influence the sensory properties of the emulsion. Emulsifiers frequently used for this purpose are soaps of stearic and/or palmitic acid, so-called triple-pressed stearic acid—a mixture of palmitic and stearic acids—generally being used in most cases as sodium, potassium or triethanolamine soap. However, it has often been found that emulsions such as these form emulsions with only limited phase stability and that the necessary viscosity can only be adjusted using high concentrations of polymers.

The water resistance of a sun protection formulation is normally achieved by the addition of polymers such as, for example, PVP/Hexadecene Copolymer. However, these polymers have the disadvantage that they seriously impair the sensory properties of the emulsion in regard to absorption behavior, spreadability and tackiness.

Accordingly, the problem addressed by the present invention was to provide cosmetic and/or pharmaceutical emulsions which would have a higher viscosity and phase stability than known commercially available products. In addition, the products would be highly water-resistant but would still be easy to spread over the skin, would be readily absorbed by the skin and would not leave it feeling tacky.

### DESCRIPTION OF THE INVENTION

The present invention relates to cosmetic and/or pharmaceutical emulsions containing long-chain hydroxyfatty acid, preferably 12-hydroxystearic acid, and/or salts thereof.

It has surprisingly been found that soaps of long-chain hydroxyfatty acids in particular, optionally in combination with the free acid, not only build up higher viscosities in emulsions than the known fatty acids, they also provide for better phase stability and improve the sensory behavior of the emulsions, i.e. are easy to spread over the skin, are readily absorbed by the skin and are non-tacky. This even applies when only part of the stearic acid is replaced by long-chain hydroxyfatty acids. The invention also includes the observation that the addition of long-chain hydroxyfatty acids or salts thereof leads to a significant improvement in the water resistance of otherwise known sun protection formulations without adversely affecting the sensory evaluation of the products.

Long-Chain Hydroxyfatty Acid and Salts Thereof

Long-chain hydroxyfatty acids in the context of the invention are understood to be hydroxyfatty acids with fatty acid chain lengths of 6 to 22, preferably 10 to 16 and more particularly 12 to 14 (long-chained) carbon atoms which contain 1 to 4, preferably 1 to 3 and more particularly 1 to 2 hydroxy groups.

Instead of the long-chain hydroxyfatty acids, preferably hydroxystearic acid and, more particularly, 12-hydroxystearic acid, salts thereof, namely the alkali metal, alkaline earth metal, ammonium, alkylammonium, alkanolammonium, glucammonium, aluminium and/or zinc salts may also be used. Long-chain hydroxyfatty acids may also be used in admixture with the salts or complete or partial neutralization may be achieved in the end formulation itself by addition of corresponding basic compounds. The long-chain hydroxyfatty acids and/or salts thereof are normally used in quantities of 0.1 to 10, preferably 0.5 to 5 and more particularly 1 to 2% by weight, based on the end formulation.

Commercial Applications

The present invention also relates to the use of long-chain hydroxyfatty acids, preferably hydroxystearic acid, and/or salts thereof for the production of waterproof sun protection preparations in which they may be present in quantities of 0.1 to 10, preferably 0.5 to 5 and more particularly 1 to 2% by weight, based on the end formulation.

The present invention also relates to the use of long-chain hydroxyfatty acids and/or salts thereof as viscosity adjusters. Where the viscosity adjusters according to the invention are used to replace or partly replace conventional viscosity adjusters, for example stearic acid, 1.3 to 10 and preferably 1.5 to 5 times higher viscosities (Brookfield RVF, spindle 5, 20 r.p.m., 23° C. after 1 week) can be measured.

Cosmetic and/or Pharmaceutical Preparations

The emulsions according to the invention may be used for creams, gels, lotions, alcoholic and aqueous/alcoholic solutions, emulsions, wax/fat compounds, stick preparations and the like and may contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, antidandruff agents, film formers, swelling agents, insect repellents, self-tanning agents, tyrosine inhibitors (depigmenting agents), hydrotropes, solubilizers, preservatives, perfume oils, dyes and the like as further auxiliaries and additives.

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to, 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₃₋₃₈ alkyl hydroxycarboxylic acids with linear or branched C₆₋₂₂ fatty alcohols (cf. DE 19756377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-/di-/triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, for example Dicaprylyl Carbonate, (Cetiol® CC), Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, for example Dicaprylyl Ether (Cetiol® OE), ring opening products of epoxidized fatty acid esters with polyols, silicone oils (cyclomethicone, silicon methicones, etc.) and/or aliphatic or naphthenic hydrocarbons such as, for example, mineral oil, Vasoline, petrolatum, squalane, squalene or dialkyl cyclohexanes.

Emulsifiers

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, C₁₂₋₂₂ fatty acids, alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alk(en)yl group and ethoxylated analogs thereof;

products of the addition of 1 to 15 mol ethylene oxide with castor oil and/or hydrogenated castor oil;

products of the addition of 15 to 60 mol ethylene oxide with castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof with 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof with 1 to 30 mol ethylene oxide;

mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethyleneglycol-30 Dipolyhydroxy-stearate;

polymer emulsifiers, for example Pemulen types (TR-1, TR-2) from Goodrich;

polyalkylene glycols and

glycerol carbonate.

Ethylene Oxide Addition Products

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations from DE 20 24 051 PS.

Alkyl and/or Alkenyl Oligoglycosides

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Partial Glycerides

Typical examples of suitable partial glycerides are long-chain hydroxyfatty acid monoglyceride, long-chain hydroxyfatty acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may also contain small quantities of triglyceride from the production prodcess. Products of the addition of 1 to 30 and preferably 5 to 10 mol ethylene oxide onto the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Anionic Emulsifiers

Typical anionic emulsifiers are salts, namely the alkali metal, alkaline earth metal, ammonium, alkylammonium, alkanolammonium, glucammonium, aluminium and/or zinc salts of aliphatic fatty acids containing 12 to 22 carbon atoms such as, for example, palmitic acid, stearic acid or behenic acid and dicarboxylic acids containing 12 to 22 carbon atoms such as, for example, azelaic acid or sebacic acid.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes, microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol(glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids containing 12 to 22 carbon atoms or 12-hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl and hydroxypropyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil., 108, 95 (1993).

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

UV Protection Factors and Antioxidants

UV protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP 0693471 B1;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzo-phenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1phenyl-3-(4′-isopropylphenyl)-propane-1,3-dione and the enamine compounds described in DE 19712033 A1 (BASF). The UV-A and UV-B filters may of course also be used in the form of mixtures. Particularly favorable combinations consist of the derivatives of benzoyl methane, for example 4-tert.butyl-4′-methoxydibenzoyl methane (Parsol® 1789) and 2-cyano-3,3-phenylcinnamic acid-2-ethylhexyl ester (Octocrylene), in combination with esters of cinnamic acid, preferably 4-methoxycinnamic acid-2-ethylhexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used. Other suitable UV filters can be found in P. Finkel's review in SÖFWV-Journal 122, 543 (1996) and in Parf. Kosm. 3, 11 (1999).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and heptathionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prune extract, bambara nut extract, and vitamin complexes.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxy-diphenylether (triclosan), 4-chloro-3,5-dimethylphenol, 2,2′methylene-bis-(6-bromo-4-chlorophenol), 3-methyl-4-(1-methylethyl)-phenol, 2-benzyl4-chlorophenol, 3-(4-chlorophenoxy)propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-noctyl amide or salicylic acid-n-decyl amide.

Enzyme Inhibitors

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation.

Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Odor Absorbers

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat.

Antiperspirants

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H )-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxyphenyl}-piperazine, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid, the silver complexes known by the name of Surfacine® and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), Woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes as listed, for example, in the publication “Kosmetische Färbemittel” of the Farbstoffkommission der Deutschen Forschungsgemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular preparation. The preparations may be produced by standard hot or cold processes and are preferably produced by the phase inversion temperature method.

### EXAMPLES OF THE USE OF 12-HYDROXYSTEARIC ACID

### Examples 1 to 3, Comparison Examples C1 and C2

The water resistance of the following formulations was determined as follows:

carrier material: Vitro-Skin N19, a product of IMS (4×3 cm), on transparency frames

quantity applied: 2 mg/cm²

drying time before 1st measurement: 15 mins., temperature 30° C.

water temperature: 23° C. (16° d)

pH value water: 7.0±0.5

water volume: 400 ml

stirring speed: 300 r.p.m. (magnetic stirrer)

watering time: 2×20 mins with a 20-minute interval

drying time before 2nd measurement: 15 mins., temperature 30° C.

The SPF is determined before and after watering using a UV-1000S Labsphere Ultraviolet Transmittance Analyzer. The results are set out in Table 1. Examples 1 to 3 correspond to the invention, Examples C1 and C2 are intended for comparison. All quantities are in % by weight. The sensory evaluation was conducted by a panel of trained volunteers who awarded scores of (1)=very good to (6)=unsatisfactory. The final result represents the average of three measurements.

### Examples 4 to 7, Comparison Examples C3 and C4

Examples 4 and 5 according to the invention show that distinctly higher viscosities and improved thermal stabilities are achieved in comparison with C3 and C4 for a comparable formulation. At the same time, sensory behavior is considerably improved. Examples 6 and 7 show that the percentage polymer content can be reduced by 50% by weight to build up comparable viscosities to C3 and C4, even when the stearic acid is only partly replaced by 12-hydroxystearic acid. All the emulsions according to the invention are stable in storage and have good sensory properties. Ca. 95% by weight of the 12-hydroxystearic acid is present as the triethanolamine salt in the Examples. Viscosity was determined with a Brookfield RVF viscosimeter (spindle 5, 20 r.p.m., 23° C. after 1 week). Stability was also determined using the school mark scale. The results are set out in Table 2.

Tables 3 and 4 contain a number of sun protection formulations while Tables 5 and 6 show examples of care emulsions according to the invention.

TABLE 1
Evaluation of sun protection formulations (quantities in % by weight
of the final concentrations)

| Composition/performance | 1 | 2 | 3 | C1 | C2 |
|---|---|---|---|---|---|
| Polyglyceryl-2 Dipolyhydroxystearate | 4.0 | 4.0 | 4.0 | 4.0 | 4.0 |
| (and) Lauryl Glucoside (and) | | | | | |
| Glycerin | | | | | |
| Cocoglycerides | 7.0 | 7.0 | 7.0 | 7.0 | 7.0 |
| Dicaprylyl Ether | 6.0 | 6.0 | 6.0 | 6.0 | 6.0 |
| Hexyldecanol | 3.0 | 3.0 | 3.0 | 3.0 | 3.0 |
| 12-Hydroxystearic acid | 2.0 | 4.0 | 1.0 | — | — |
| 12-Hydroxystearic acid sodium salt | — | — | 2.0 | — | — |
| PVP/Hexadecene Copolymer | — | — | — | 2.0 | 4.0 |
| Octyl Methoxycinnamate | 7.5 | 7.5 | 7.5 | 7.5 | 7.5 |
| Butyl Methoxydibenzoylmethane | 2.0 | 2.0 | 2.0 | 2.0 | 2.0 |
| Polyacrylate | 0.2 | 0.2 | 0.2 | 0.2 | 0.2 |
| Glycerin | 5.0 | 5.0 | 5.0 | 5.0 | 5.0 |
| Water, NaOH, preservative | to 100, pH = 7 | | | | |
| In vitro sun protection factor (SPF) | | | | | |
| before the treatment with water | 15 | 15 | 15 | 15 | 15 |
| after the treatment with water | 12 | 14 | 13 | 9 | 11 |
| difference (%-rel) | 80 | 93 | 87 | 60 | 73 |
| Sensory evaluation | | | | | |
| Absorption | 1 | 2 | 1 | 4 | 6 |
| Smoothness | 1 | 2 | 1 | 4 | 6 |
| Tackiness | 1 | 2 | 1 | 5 | 6 |

TABLE 2
Evaluation of the viscosity and stability of cosmetic and/or
pharmaceutical emulsions-quantities in % by weight of the
final concentrations

| Composition/ | | | | | | |
|---|---|---|---|---|---|---|
| performance | 4 | C3 | 5 | C4 | 6 | 7 |
| Stearic acid, triple- | — | 3 | — | 3 | — | 1.5 |
| pressed | | | | | | |
| 12-Hydroxystearic acid | 3 | — | 3 | — | 3 | 1.5 |
| Paraffin oil | 16 | 16 | — | — | — | — |
| Cocoglycerides | — | — | 16 | 16 | 16 | 16 |
| Triethanolamine | 1.4 | 1.4 | 1.4 | 1.4 | 1.4 | 1.4 |
| Carbopol 2050, 2% by | 10 | 10 | 10 | 10 | 5 | 5 |
| weight swelling | | | | | | |
| Water, preservative | to 100 | | | | | |
| Viscosity and stability | | | | | | |
| Viscosity [mPas] | 13,200 | 4,900 | 14,600 | 3,700 | 5,500 | 5,000 |
| Stability after 1 w, | 1 | 6 | 1 | 6 | 1 | 2 |
| 45° C. | | | | | | |
| Sensory evaluation | | | | | | |
| Absorption | 1 | 5 | 1 | 5 | 1 | 2 |
| Smoothness | 2 | 6 | 1 | 5 | 1 | 2 |
| Tackiness | 1 | 6 | 1 | 5 | 1 | 2 |

TABLE 3
O/W sun protection emulsions-quantities in % by weight of the final concentrations

| Component (INCI) | 1 | 2 | 3 | 4 | 5 | 6 | 7 | 8 | 9 | 10 | 11 |
|---|---|---|---|---|---|---|---|---|---|---|---|
| L = Lotion, C = Cream | L | C | C | L | C | L | L | C | L | C | L |
| Eumulgin VL 75 (Polyglyceryl-2 | | | | | | 4 | 4 | | | | |
| Dipolyhydroxystearate, Lauryl Glucoside, Glycerin) | | | | | | | | | | | |
| Eumulgin B2 (Ceteareth-20) | 2 | | | | | | | | | | |
| Polysorbate 60 | | | | 1 | | | | | | | |
| PEG-30 Stearate | | 3 | | 2 | | | | | | | |
| Cutina E 24 (PEG-20 Glyceryl Stearate) | 1 | | | | | | | | | | |
| Hostaphat KL 340 N (Trilaureth-4 Phosphate) | | | | | | | | | 2 | | |
| Lanette E (Sodium Cetearyl Sulfate) | | | | | | | | | | | |
| Amphisol K (Potassium Cetyl Phosphate) | | | 1 | | | 1 | | | | 1 | |
| Sodium Stearate | | | | | | | 1 | | | | |
| Emulgade PL 68/50 (Cetearyl Glucoside, Cetearyl | | | 5 | | 5 | | | | | 4 | |
| Alcohol) | | | | | | | | | | | |
| TegoCare 450 (Polyglyceryl-3 Methylglucose | | | | | | | | | | 3 | |
| Distearate) | | | | | | | | | | | |
| Cutina MD (Glyceryl Stearate) | 4 | | 4 | 6 | | | 4 | | | 6 | |
| Lanette 14 (Myristyl Alcohol) | 1 | | | 1 | | | | 2 | | | 4 |
| Lanette O (Cetearyl Alcohol) | 1 | 6 | | | 5 | 2 | | | | | |
| 12-Hydroxystearic Acid | 2 | 2 | 4 | 1 | 2 | 2 | 2 | 4 | 2 | 4 | 1 |
| Sodium 12-Hydroxystearate | | | | | 2 | | | 2 | | | 2 |
| Lanolin Alcohol | | | | | .5 | .5 | | | | | |
| Lanolin | | | | | | | 5 | | | | |
| Myritol PC (Propylene Glycol | | | | | 5 | | | | | | |
| Dicaprylate/Dicaprate) | | | | | | | | | | | |
| Myritol 331 (Cocoglycerides) | 5 | | | | | 6 | | 12 | | | |
| Finsolv TN (C12/15 Alkyl Benzoate) | | | 3 | | | | | | 8 | | |
| Cetiol CC (Dicaprylyl Carbonate) | | 2 | | | | 4 | 4 | | | | |
| Cetiol OE (Dicaprylyl Ether) | | | 3 | | | | | | 2 | 3 | |
| DC 244 (Cyclomethicone) | 4 | | 1 | | 5 | | | | | | 2 |
| DC 2502 (Cetyl Dimethicone) | | 1 | | | 2 | | | | | | |
| Hydrogenated Polyisobutene | | | | | | | 4 | | | | |
| Dimethicone | | 2 | | | | | | | | | |
| Cetiol 868 (Ethylhexyl Stearate) | | | 2 | | 2 | | 4 | | | | 7 |
| Cetiol J 600 (Oleyl Erucate) | | | | | 3 | 2 | | | | 5 | |
| Mineral Oil | | | | 9 | | | | | | | |
| Cetiol B (Butyl Adipate) | | | 1 | | | | | | | 2 | |
| Eutanol G (Octyldodecanol) | | | | | | | | | | | |
| Eutanol G 16 (Hexyldecanol) | | | | | | | | | | | |
| Cetiol PGL (Hexyldecanol, Hexyldecyl Laurate) | | 5 | | | | | | | | 5 | |
| Almond Oil | | | 2 | | | | 1 | | | | |
| Photonyl LS | | | | 2 | | | | | | 2 | |
| Panthenol | 1 | | | | | | | | | | |
| Bisabolol | 0.2 | | | | | | | | | | |
| Tocopherol/Tocopheryl Acetate | 1 | | | | | | | | | | |
| Photonyl LS | | | | | | | | | | | |
| Phenylbenzimidazole Sulfonic Acid (Sodium Salt) | 2 | | 2.2 | | 3 | 3 | | | | | 2 |
| Octocrylene | 3 | 5 | | 4 | | | | | | | |
| Benzophenone-3 | | | | | 1 | | | | | | 2 |
| 4-Methylbenzylidene Camphor | 2 | | 3 | 3 | | 2 | 2 | 2 | | | 1 |
| Octyl Salicylate | | | | | | | | | 10 | 7 | |
| Isoamyl p-Methoxycinnamate | | 7.5 | 6 | 6 | | | | 5 | | | 6 |
| Octyl Methoxcinnamate | | | | | | 7.5 | 4 | 5 | | | |
| Octyl Triazone | 2 | | | | 2.5 | | | 1 | | | |
| Butyl Methoxydibenzoylmethane | | 1 | 1 | | | | 2 | | 2 | 2 | |
| Zinc Oxide (micronized and/or surface-treated) | 10 | | | | | 10 | | | | 5 | 4 |
| Titanium Dioxide (micronized and/or surface-treated) | | | 10 | | 5 | | 3 | 6 | | | 4 |
| Magnesium Aluminium Silicates (e.g. Veegum types) | | | 1 | | | | | | 1 | | |
| Xanthan Gum (e.g. Keltrol types) | | | .5 | | | | | | .5 | | |
| Carbomer (e.g. Carbopol and/or Pemulen types) | | .5 | | .2 | .2 | .2 | | .5 | .1 | .3 | .2 |
| Ethanol | | | | | | | | | | 10 | |
| Butylene Glycol | | 2 | | 4 | 3 | | 2 | 5 | 2 | | |
| Glycerin | 5 | 5 | 5 | | 3 | 3 | 2 | | 4 | | 3 |

| Water, preservative, NaOH | to 100 | | | | | | | | | | |
|---|---|---|---|---|---|---|---|---|---|---|---|
| Component (INCI) | 12 | 13 | 14 | 15 | 16 | 17 | 18 | 19 | 20 | 21 | 22 |
| L = Lotion, C = Cream | C | L | L | C | L | C | L | L | L | L | C |
| Eumulgin VL 75 (Polyglyceryl-2 | 4 | 3 | | | 3 | | | | | | |
| Dipolyhydroxystearate, Lauryl Glucoside, Glycerin) | | | | | | | | | | | |
| Eumulgin B2 (Ceteareth-20) | | | | | | | | | | 1 | |
| Polysorbate 60 | | | | | | | | | | 1 | |
| PEG-30 Stearate | | | | | | | | | | | |
| Cutina E 24 (PEG-20 Glyceryl Stearate) | | | | 2 | | | | | | | |
| Hostaphat KL 340 N (Trilaureth-4 Phosphate) | | | | | | | | | | .5 | |
| Lanette E (Sodium Cetearyl Sulfate) | | | | .5 | | | | | .5 | | |
| Amphisol K (Potassium Cetyl Phosphate) | | | | | | 1 | 1 | 1 | | | |
| Sodium Stearate | | | | | 1 | | | | | | |
| Emulgade PL 68/50 (Cetearyl Glucoside, Cetearyl | | 6 | | | | 4.5 | 2.5 | 5 | | | |
| Alcohol) | | | | | | | | | | | |
| TegoCare 450 (Polyglyceryl-3 Methylglucose | 5 | | | | | | | | 4 | | |
| Distearate) | | | | | | | | | | | |
| Cutina MD (Glyceryl Stearate) | 3 | | | 8 | 6 | 1 | | | | 4 | 1 |
| Lanette 14 (Myristyl Alcohol) | | 2 | | | | | | 2 | | 1 | |
| Lanette O (Cetearyl Alcohol) | | | | 2 | | | | | 1 | 1 | |
| 12-Hydroxystearic Acid | 2 | 2 | 1 | 1 | 1 | 4 | 2 | 2 | 2 | 1 | 3 |
| Sodium 12-Hydroxystearate | 1 | 1 | 1 | 2 | 3 | | | 1 | | 2 | 1 |
| Lanolin Alcohol | | | | | | | | | | | |
| Lanolin | | | | | | | | | | | |
| Myritol PC (Propylene Glycol | 6 | | | | | | | | 5 | | |
| Dicaprylate/Dicaprate) | | | | | | | | | | | |
| Myritol 331 (Cocoglycerides) | 5 | | 12 | | | 8 | 8 | | | 10 | 8 |
| Finsolv TN (C12/15 Alkyl Benzoate) | | | | | 5 | | | 3 | 3 | | |
| Cetiol CC (Dicaprylyl Carbonate) | | | 6 | | | 5 | 5 | | | | |
| Cetiol OE (Dicaprylyl Ether) | | | | | 2 | | | | | | 2 |
| DC 244 (Cyclomethicone) | | 2 | | | 1 | | | | | | |
| DC 2502 (Cetyl Dimethicone) | | 1 | | | 1 | | | | | | |
| Hydrogenated Polyisobutene | | | | | | | | | | | |
| Dimethicone | | | | | 1 | | | | | | |
| Cetiol 868 (Ethylhexyl Stearate) | | 2 | | | | | | | | | |
| Cetiol J 600 (Oleyl Erucate) | | 2 | | | | | | | | | |
| Mineral Oil | | | | 10 | | | | | | | |
| Cetiol B (Butyl Adipate) | | | 4 | | | | | 4 | | | |
| Eutanol G (Octyldodecanol) | | 3 | | | | | | | | | |
| Eutanol G 16 (Hexyldecanol) | | | | | | | | | | | 3 |
| Cetiol PGL (Hexyldecanol, Hexyldecyl Laurate) | | | | | | | | | 2 | | |
| Almond Oil | | | | | | | | | | | |
| Photonyl LS | | | | | | | | | | 2 | |
| Panthenol | 1 | | | | | | | | | | |
| Bisabolol | 0.2 | | | | | | | | | | |
| Tocopherol/Tocopheryl Acetate | 1 | | | | | | | | | | |
| Photonyl LS | | | | | | | | | | | |
| Phenylbenzimidazole Sulfonic Acid (Sodium Salt) | | | | | | | | | | 3 | |
| Octocrylene | 6 | | 9 | | 5 | | | | 4 | | 7 |
| Benzophenone-3 | | | | | | | | | | | |
| 4-Methylbenzylidene Camphor | | 2 | | 1 | | | | 3 | | | 3 |
| Octyl Salicylate | | | | | | | | | 7 | | |
| Isoamyl p-Methoxycinnamate | | 4 | | | | | | 5 | | | |
| Octyl Methoxcinnamate | | 4 | 7.5 | 5 | | | | 5 | | 7.5 | |
| Octyl Triazone | 1 | | | | | | | | 2.3 | | |
| Butyl Methoxydibenzoylmethane | | | | | | | | | 2 | | |
| Zinc Oxide (micronized and/or surface-treated) | | | 5 | | | 5 | 5 | | 6 | | 7 |
| Titanium Dioxide (micronized and/or surface-treated) | | | | | 10 | 7 | 7 | 10 | | | |
| Magnesium Aluminium Silicates (e.g. Veegum types) | | | 1.5 | | | 1.5 | 1.2 | | 1 | | 1.5 |
| Xanthan Gum (e.g. Keltrol types) | | | .5 | | | .5 | .4 | | .5 | | .5 |
| Carbomer (e.g. Carbopol and/or Pemulen types) | | .3 | | .3 | | | | .2 | | | |
| Ethanol | | 5 | | 8 | | | | | | | |
| Butylene Glycol | 5 | | | 3 | 3 | | | | | 8 | |
| Glycerin | 2 | 4 | 3 | 3 | | 3 | 3 | 3 | 5 | | 3 |

| | |
|---|---|
| Water, preservative, NaOH | to 100 |

TABLE 4
W/O sun protection emulsions-quantities in % by weight of the final concentrations

| Component (INCI) | 1 | 2 | 3 | 4 | 5 | 6 | 7 | 8 | 9 | 10 | 11 |
|---|---|---|---|---|---|---|---|---|---|---|---|
| L = Lotion, C = Cream | C | L | C | L | C | L | L | L | L | C | C |
| Dehymuls PGPH (Polyglyceryl-2 Dipolyhdroxystearate) | 4 | 2 | | 3 | 3 | | | 2 | 2 | 4 | |
| Monomuls 90-O18 (Glyceryl Oleate) | | | 2 | | | | | | | | |
| Lameform TGI (Polyglyceryl-3 Diisostearate) | 2 | | 4 | | 3 | | | | | 1 | 3 |
| Abil EM 90 Cetyl (Dimethicone Copolyol) | | | | | | | 4 | | | | |
| Glucate DO (Methyl Glucose Dioleate) | | | | | | | | | | | 3 |
| Isolan PDI (Diisostearoyl Polyglyceryl-3 Diisostearate) | | | | | | 4 | | 2 | | | |
| Sorbitan Sesquioleate | | | | 2 | | | | | | | |
| Elfacos ST9 (PEG-45/Dodecyl Glycol Copolymer) | | | | | | | | | 2 | | |
| Elfacos ST37 (PEG-22/Dodecyl Glycol Copolymer) | | | | | | | | | | | |
| Arlacel P 135 (PEG-30 Dipolyhydroxystearate) | | 2 | | | | | | | | | |
| Dehymuls HRE 7 (PEG-7 Hydrogenated Castor Oil) | | | | | | | | | | | |
| Zinc Stearate | 1 | | | 1 | 1 | | | 1 | | 1 | |
| Microcrystalline Wax | | | 5 | | | 2 | | | | | 5 |
| Beeswax | 3 | | | 1 | | | | 5 | | 7 | |
| Tego Care CG (Cetearyl Glucoside) | | | | | 1 | | | | | | .5 |
| Isostearic Acid | | | 1 | 1 | | 1 | 1 | | | | 1 |
| 12-Hydroxystearic Acid | 3 | 3 | 2 | 1 | 1 | 2 | | 2 | | 1 | 1 |
| Sodium 12-Hydroxystearate | | 1 | | 4 | 3 | 2 | 4 | | 3 | 2 | 1 |
| Lanolin | | | 5 | | | | | | | 4 | |
| Lanolin Alcohol | | | | | | | | | | | 1 |
| Myritol PC (Propylene Glycol Dicaprylate/Dicaprate) | | | | | 3 | | | 4 | | | |
| Myritol 331 (Cocoglycerides) | 10 | | | | 3 | 6 | | | | | 8 |
| Finsolv TN (C12/15 Alkyl Benzoate) | | | | 5 | | | 5 | | | | |
| Cetiol CC (Dicaprylyl Carbonate) | 12 | 4 | | | | 2 | | | 2 | | 5 |
| Cetiol OE (Dicaprylyl Ether) | | | | | 4 | | 5 | | 4 | 2 | |
| DC 244 (Cyclomethicone) | | 3 | | | | | 2 | | | | |
| DC 2502 (Cetyl Dimethicone) | | | 1 | | 2 | | | | | | |
| Hydrogenated Polyisobutene | | | | | | | | | | 10 | |
| Dimethicone | | | | 4 | | | | 3 | | | |
| Cetiol 868 (Ethylhexyl Stearate) | | | | | | | | | | 2 | |
| Eutanol G 16 (Hexyldecanol) | | | | | | | | | | | |
| Eutanol G 16S (Hexyldecyl Stearate) | | | | | | | | | | | |
| Cetiol J 600 (Oleyl Erucate) | | | 4 | | | 2 | | | | | |
| Ceraphyl 45 (Dioctyl Malate) | | | | 2 | | | | 2 | | 6 | |
| Mineral Oil | | | | | 4 | | | | | | |
| Cetiol B (Butyl Adipate) | | | 2 | 4 | | | | | | 3 | |
| Eutanol G (Octyldodecanol) | | | 3 | | | | | 8 | | | |
| Cetiol PGL (Hexyldecanol, Hexyldecyl Laurate) | | 11 | | | | 4 | | | 9 | | |
| Ceraphyl 45 (Dioctyl Malate) | | | | 2 | | | | 2 | | 6 | |
| Almond Oil | | | | | 1 | | 5 | | | | |
| Photonyl LS | | 2 | | 1 | | | | | 4 | | |
| Panthenol | 1.0 | | | | | | | | | | |
| Bisabolol | 0.2 | | | | | | | | | | |
| Tocopherol/Tocopheryl Acetate | 1.0 | | | | | | | | | | |
| Magnesium sulfate x 7 water | 1 | | | | | | | | | | |
| Phenylbenzimidazole Sulfonic Acid (Sodium Salt) | | 1 | | 3 | | | | | | | |
| Octocrylene | | | | | 4 | | | | | 6 | |
| Benzophenone-3 | | | 2 | | | | 2 | | | | |
| 4-Methylbenzylidene Camphor | | 3 | | | | | | 4 | | 3 | |
| Octyl Salicylate | | | | | | | | | | | |
| Isoamyl p-Methoxycinnamate | | 7.5 | | | | | | | 5 | | |
| Octyl Methoxycinnamate | | | 6 | 6 | | 7.5 | 7.5 | | 5 | | 7.5 |
| Octyl Triazone | | | | | 2.5 | | | 1 | | 2 | |
| Butyl Methoxydibenzoylmethane | | | | | | | | 1 | | 2 | |
| Zinc Oxide (micronized and/or surface-treated) | | | | | | 6 | | | | | |
| Titanium Dioxide (micronized and/or surface-treated) | 15 | | 10 | | 5 | | 4 | | | | 4 |
| Ethanol | | | | | | | | | | 8 | |
| Butylene Glycol | | | 2 | 6 | | | 2 | 5 | | | 2 |
| Glycerin | 5 | 3 | 3 | | 5 | 3 | 2 | | 10 | 4 | |

| Water, preservative | to 100 | | | | | | | | | | |
|---|---|---|---|---|---|---|---|---|---|---|---|
| Component (INCI) | 12 | 13 | 14 | 15 | 16 | 17 | 18 | 19 | 20 | 21 | 22 |
| L = Lotion, C = Cream | L | C | L | L | C | L | L | L | L | C | C |
| Dehymuls PGPH (Polyglyceryl-2 Dipolyhdroxystearate) | 3 | | 5 | | | 3 | | 4 | | | 4 |
| Monomuls 90-O18 (Glyceryl Oleate) | | 1 | | | | | | | | | |
| Lameform TGI (Polyglyceryl-3 Diisostearate) | | | | | 4 | | | 1 | | 3 | 1 |
| Abil EM 90 Cetyl (Dimethicone Copolyol) | | | | 1 | | | | | | 2 | |
| Glucate DO (Methyl Glucose Dioleate) | | | | 3 | | | | | 2 | | |
| Isolan PDI (Diisostearoyl Polyglyceryl-3 Diisostearate) | | 3 | | | | | 4 | | | | |
| Sorbitan Sesquioleate | | | | | | 3 | | | | | |
| Elfacos ST9 (PEG-45/Dodecyl Glycol Copolymer) | | | | | | | | | | | 2 |
| Elfacos ST37 (PEG-22/Dodecyl Glycol Copolymer) | 2 | | | | | | | | | | |
| Arlacel P 135 (PEG-30 Dipolyhydroxystearate) | | | | | | 3 | | | | | |
| Dehymuls HRE 7 (PEG-7 Hydrogenated Castor Oil) | | | | | | | | | 4 | | |
| Zinc Stearate | | 2 | 2 | 1 | 1 | | | 1 | 1 | | |
| Microcrystalline Wax | | | | | 4 | | 1 | | | 4 | |
| Beeswax | | 4 | | 2 | | | 1 | 1 | 2 | | 1 |
| Tego Care CG (Cetearyl Glucoside) | | | | | | | | | | | |
| Isostearic Acid | 1 | 1 | | | | | 1 | 1 | | 1 | 1 |
| 12-Hydroxystearic Acid | 2 | 4 | 3 | | 2 | 2 | 1 | 3 | 3 | 1 | 4 |
| Sodium 12-Hydroxystearate | | | 1 | 4 | | 2 | 4 | | 1 | 2 | |
| Lanolin | | 7 | 3 | | | | | | | | |
| Lanolin Alcohol | | | | | | | | | | | |
| Myritol PC (Propylene Glycol Dicaprylate/Dicaprate) | | | | | | | | | | | |
| Myritol 331 (Cocoglycerides) | 4 | | 3 | | 5 | | | | 5 | 4 | |
| Finsolv TN (C12/15 Alkyl Benzoate) | | | 5 | | | 7 | | | | | |
| Cetiol CC (Dicaprylyl Carbonate) | 3 | | | | | | 3 | | | | 12 |
| Cetiol OE (Dicaprylyl Ether) | | 3 | | 2 | | | 3 | | | | |
| DC 244 (Cyclomethicone) | | 4 | | 2 | | | | | | | |
| DC 2502 (Cetyl Dimethicone) | | | | 1 | | | | | | | |
| Hydrogenated Polyisobutene | | | | | | | | | | | |
| Dimethicone | | | | 1 | | | | 4 | | | |
| Cetiol 868 (Ethylhexyl Stearate) | | | | | | | | | | | |
| Eutanol G 16 (Hexyldecanol) | | | | | | | | | | | 3 |
| Eutanol G 16S (Hexyldecyl Stearate) | | | | | | | | | | | 7 |
| Cetiol J 600 (Oleyl Erucate) | | | | 3 | | | | | | | |
| Ceraphyl 45 (Dioctyl Malate) | | | | 1 | | | | | 5 | 4 | |
| Mineral Oil | | | | | | | 9 | | | | |
| Cetiol B (Butyl Adipate) | | | | | 3 | 3 | | 2 | 2 | | |
| Eutanol G (Octyldodecanol) | | | | 2 | | | | | 5 | | |
| Cetiol PGL (Hexyldecanol, Hexyldecyl Laurate) | | | | | | | | 6 | | | |
| Ceraphyl 45 (Dioctyl Malate) | | | | 1 | | | | | 5 | 4 | |
| Almond Oil | | | 2 | | | | | | | | |
| Photonyl LS | | | | | | | 3 | | | | 12 |
| Panthenol | 1.0 | | | | | | | | | | |
| Bisabolol | 0.2 | | | | | | | | | | |
| Tocopherol/Tocopheryl Acetate | 1.0 | | | | | | | | | | |
| Magnesium sulfate x 7 water | 1 | | | | | | | | | | |
| Phenylbenzimidazole Sulfonic Acid (Sodium Salt) | | 4 | | | | | | 4 | | | |
| Octocrylene | 6 | | | | | | | | 6 | | |
| Benzophenone-3 | | | | 2 | | 2 | | | | | |
| 4-Methylbenzylidene Camphor | 2 | | | | 3 | | 4 | | 2 | | |
| Octyl Salicylate | | | | | 10 | | 8 | | | | |
| Isoamyl p-Methoxycinnamate | | | 5 | 6 | | | | | | 5 | |
| Octyl Methoxycinnamate | | 5 | 5 | | | 7.5 | | | | 5 | |
| Octyl Triazone | 1 | | | 2 | 2 | | | | 3 | 2 | |
| Butyl Methoxydibenzoylmethane | | | 1 | | | | 1 | | 0.5 | | |
| Zinc Oxide (micronized and/or surface-treated) | 4 | 10 | | | | | | | | | 5 |
| Titanium Dioxide (micronized and/or surface-treated) | | | | 2 | | 3 | | 10 | | 4 | 7 |
| Ethanol | | | | 8 | | 10 | | | | | |
| Butylene Glycol | 5 | 1 | | 3 | 3 | | | | 8 | 2 | |
| Gtrcerin | | | 6 | 2 | | | 5 | 5 | | 3 | 5 |

| | |
|---|---|
| Water, preservative | to 100 |

TABLE 5
W/O care emulsions-quantities in % by weight of the final concentrations

| Component (INCI) | 1 | 2 | 3 | 4 | 5 | 6 | 7 | 8 | 9 | 10 | 11 |
|---|---|---|---|---|---|---|---|---|---|---|---|
| L = Lotion, C = Cream | C | L | C | L | C | L | L | L | C | C | C |
| Na-, K-, Zn-, Mg-, Al-12-Hydroxysterate | 1 | 1 | 2 | 1 | 1 | 1 | 1 | 1 | 2 | 2 | 1 |
| 12-Hydroxystearic Acid | 2 | | 3 | | 1 | | | | 1 | 2 | |
| Dehymuls PGPH (Polyglyceryl-2 Dipolyhydroxystearate) | | 3 | | | 3 | | | | | | |
| Monomuls 90-O18 (Glyceryl Oleate) | 2 | | | | | | | | 2 | | 2 |
| Lameform TGI (Polyglyceryl-3 Diisostearate) | 4 | 1 | | | 3 | | | 1 | 4 | 3 | 3 |
| Abil EM 90 (Cetyl Dimethicone Copolyol) | | | | | | | 4 | | | | |
| Isolan PDI (Diisostearoyl Polyglyceryl-3 Diisostearate) | | | | | | 4 | | | | | |
| Glucate DO (Methyl Glucose Dioleate) | | | | 5 | | | | | | | |
| Sorbitan Sesquioleate | | | 5 | | | | | | | | |
| Dehymuls FCE (Dicocoyl Pentaerythrityl Distearyl | | | | | | | | | | | |
| Citrate) | | | | | | | | | | | |
| Dehyymuls HRE 7 (PEG-7 Hydrogenated Castor Oil) | | | | | | | | 4 | | 1 | |
| Zinc Stearate | 2 | 1 | | 1 | 1 | | | 1 | 1 | 1 | |
| Microcrystalline Wax | | | 5 | | | 2 | | | | | 5 |
| Beeswax | 4 | | | 1 | | | | 1 | 4 | 7 | |
| Cetearyl Glucoside (Tego Care CG) | | | | | 1 | | | | | | .5 |
| Isostearic Acid | | | 1 | 1 | | 1 | 1 | | | | 1 |
| Dry Flo Plus (Aluminium Starch Octenyylsuccinate) | | | | | | | | | | | |
| SFE 839 (Cyclopentasiloxan, | | | | | | | 3 | | | | |
| Dimethicone/Vinyldimethicone Crosspolymer) | | | | | | | | | | | |
| Lanolin Alcohol | 1 | | | | | | | | | | 1 |
| Lanolin | | | 5 | | | | | | | 4 | |
| Cegesoft C 17 (Myristyl Lactate) | | | 3 | | | | | | | 1 | |
| Myritol PC (Propylene Glycol Dicaprylate/Dicaprate) | | | | | 3 | | | 4 | | | |
| Myritol 331 (Cocoglycerides) | 6 | | | | | 6 | | | | | 8 |
| Finsolv TN (C12/15 Alkyl Benzoate) | | | | 5 | | | 5 | | | | |
| Cetiol A (Hexyl Laurate) | | 6 | | | | 4 | | | | | |
| Cetiol CC (Dicaprylyl Carbonate) | | 8 | | | | 2 | | | | | 5 |
| Cetiol SN (Cetearyl Isononanoate) | | 5 | | | | | | 33 | | | |
| Cetiol OE (Dicaprylyl Ether) | 3 | | | | 4 | | 5 | | 4 | 2 | |
| DC 244 (Cyclomethicone) | | | | | | | 2 | | | | |
| DC 2502 (Cetyl Dimethicone) | | | 1 | | 2 | | | | | | |
| Hydrogenated Polyisobutene | | | | | 3 | | | | | | |
| Dimethicone | | | | 4 | | | | 3 | | | |
| Cetiol 868 (Octyl Stearate) | | | | | | | | | | 2 | 7 |
| Cetiol J 600 (Oleyl Erucate) | | | 4 | | | 2 | | | | | |
| Ceraphyl 45 (Dioctyl Malate) | | | | 2 | | | | 2 | | 6 | |
| Mineral Oil | | | | | 4 | | | | | | |
| Cetiol B (Butyl Adiate) | | | 2 | 4 | | | | | | 3 | |
| Eutanol G 16 (Hexyldecanol) | | 1 | | | | | | | | 3 | |
| Eutanol G (Octyldodecanol) | | | 3 | | | | | 8 | | | |
| Cetiol PGL (Hexyldecanol (and) Hexyldecyllaurate) | | | | | | 4 | | | 9 | | |
| Almond Oil | | | | | 1 | | 5 | | | | |
| Photonyl LS | 2 | 2 | | | | | | | | | |
| Panthenol | 1.0 | | | | | | | | | | |
| Bisabolol | 0.2 | | | | | | | | | | |
| Tocopherol/Tocopheryl Acetate | 1.0 | | | | | | | | | | |
| Magnesium sulfate x 7 water | 1 | | | | | | | | | | |
| Quaternium 18-Hectorite | | | | | 1 | | | | | | |
| Propylene Carbonate | | | | | .5 | | | | | | |
| Ethanol | | | | | | | | | | 8 | |
| Butylene Glycol | | | 2 | 6 | | | 2 | 5 | | | 2 |
| Glycerin | 5 | 3 | 3 | | 5 | 3 | 2 | | 10 | 4 | |
| Water, preservative | to 100 | | | | | | | | | | |
| Component (INCI) | 12 | 13 | 14 | 15 | 16 | 17 | 18 | 19 | 20 | 21 | 22 |
| L = Lotion, C = Cream | L | C | L | L | C | L | L | L | L | C | C |
| Na-, K-, Zn-, Mg-, Al-12-Hydroxysterate | 1 | 3 | 1 | 2 | 1 | 2 | 1 | 1 | 1 | 1 | 3 |
| 12-Hydroxystearic Acid | | 1 | | | | | | | | | 1 |
| Dehymuls PGPH (Polyglyceryl-2 Dipolyhydroxystearate) | 3 | | 5 | | | 3 | 3 | 4 | | | |
| Monomuls 90-O18 (Glyceryl Oleate) | | 1 | | 1 | | | | | | | |
| Lameform TGI (Polyglyceryl-3 Diisostearate) | | | | | 4 | | | 1 | | 3 | |
| Abil EM 90 (Cetyl Dimethicone Copolyol) | | | | 3 | | | | | | 2 | |
| Isolan PDI (Diisostearoyl Polyglyceryl-3 Diisostearate) | | 3 | | | | | | | | | 4 |
| Glucate DO (Methyl Glucose Dioleate) | 1 | | | | | | | | | | |
| Sorbitan Sesquioleate | | | | | | 3 | | | | | |
| Dehymuls FCE (Dicocoyl Pentaerythrityl Distearyl | | | | | 4 | | 1 | | | | |
| Citrate) | | | | | | | | | | | |
| Dehyymuls HRE 7 (PEG-7 Hydrogenated Castor Oil) | | | | | | | | | 7 | | |
| Zinc Stearate | | 2 | 2 | 1 | 1 | 1 | | 1 | 1 | | 1 |
| Microcrystalline Wax | | | | | 4 | | 1 | | | 4 | |
| Beeswax | | 4 | | 2 | | 2 | 1 | 1 | 2 | | 5 |
| Cetearyl Glucoside (Tego Care CG) | | | | | | | | | | | |
| Isostearic Acid | 1 | 1 | | | | | 1 | 1 | | 1 | 1 |
| Dry Flo Plus (Aluminium Starch Octenyylsuccinate) | 1 | | | | | | | | | | |
| SFE 839 (Cyclopentasiloxan, | | 5 | | | | 4 | | | | | |
| Dimethicone/Vinyldimethicone Crosspolymer) | | | | | | | | | | | |
| Lanolin Alcohol | | | | | | | | | | | |
| Lanolin | | 7 | 3 | | | | | | | | |
| Cegesoft C 17 (Myristyl Lactate) | | 2 | | | | | | | | | |
| Myritol PC (Propylene Glycol Dicaprylate/Dicaprate) | | | | 8 | | | | | | | |
| Myritol 331 (Cocoglycerides) | 4 | | 3 | | 5 | | | | 5 | 4 | |
| Finsolv TN (C12/15 Alkyl Benzoate) | | | 5 | | | 7 | | | | | |
| Cetiol A (Hexyl Laurate) | | | | | | | | 6 | | | |
| Cetiol CC (Dicaprylyl Carbonate) | 3 | | | 6 | | | 3 | | | 8 | |
| Cetiol SN (Cetearyl Isononanoate) | | | | | 5 | | | | | | |
| Cetiol OE (Dicaprylyl Ether) | | 3 | | 2 | | | 3 | | | | 8 |
| DC 244 (Cyclomethicone) | | 4 | | 2 | | | | | | | |
| DC 2502 (Cetyl Dimethicone) | | | | 1 | | | | | | | |
| Hydrogenated Polyisobutene | | | | | | | | | | | |
| Dimethicone | | | | 1 | | | | 4 | | | |
| Cetiol 868 (Octyl Stearate) | | | | | | | | | | | 10 |
| Cetiol J 600 (Oleyl Erucate) | 4 | | | 3 | | | | | | | |
| Ceraphyl 45 (Dioctyl Malate) | | | | 1 | | | | | 5 | 4 | |
| Mineral Oil | | | | | | | 9 | | | | |
| Cetiol B (Butyl Adiate) | | | | | 3 | 3 | | 2 | 2 | | |
| Eutanol G 16 (Hexyldecanol) | | | | | | | | | | | |
| Eutanol G (Octyldodecanol) | | | | 2 | | | | | 5 | | |
| Cetiol PGL (Hexyldecanol (and) Hexyldecyllaurate) | | | 10 | | | | | 6 | | | 3 |
| Almond Oil | | | 2 | | 5 | | 2 | | | | |
| Photonyl LS | | | | 2 | | | | | | | 2 |
| Panthenol | 1.0 | | | | | | | | | | |
| Bisabolol | 0.2 | | | | | | | | | | |
| Tocopherol/Tocopheryl Acetate | 1.0 | | | | | | | | | | |
| Magnesium sulfate x 7 water | 1 | | | | | | | | | | |
| Quaternium 18-Hectorite | | | | | | | | | | | |
| Propylene Carbonate | | | | | | | | | | | |
| Ethanol | | | | 8 | | 10 | | | | | |
| Butylene Glycol | 5 | 1 | | 3 | 3 | | | | 8 | 2 | 1 |
| Glycerin | | | 6 | 2 | | | 5 | 5 | | 3 | 5 |

| | |
|---|---|
| Water, preservative | to 100 |

TABLE 6
O/W care emulsions-quantities in % by weight of the final concentrations

| Component (INCI) | 1 | 2 | 3 | 4 | 5 | 6 | 7 | 8 | 9 | 10 | 11 |
|---|---|---|---|---|---|---|---|---|---|---|---|
| L = Lotion, C = Cream | C | C | C | L | C | L | L | C | L | C | L |
| Sodium/Potassium 12-Hydroxysterate | 2 | 2 | 1 | 1 | 1 | 1 | 2 | 3 | 2 | 2 | 1 |
| 12-Hydroxystearic Acid | 2 | | 3 | | | | | | | 2 | |
| Eumulgin VL 75 (Polyglyceryl-2 Dipolyhydroxystearate, | | | | | | 4 | | | | | |
| Lauryl Glucoside, Glycerin) | | | | | | | | | | | |
| Dehymuls PGPH (Polyglyceryl-2 | | 2 | | | | | | | | | |
| Dipolyhydroxystearate) | | | | | | | | | | | |
| Generol R (Rapeseed Sterols) | | | 1 | | | | | | | | |
| Eumulgin B2 (Ceteareth-20 | | | .8 | | | | | | | | |
| Polysorbate 60 | | | | 1 | | | | | | | |
| Cutina E 24 (PEG-20 Glyceryl Stearate) | | | .6 | 2 | | | | | | | |
| Hostaphat KL 340 N (Trilaureth-4 Phosphate) | | | | | | | | | 2 | | |
| Lanette E (Sodium Cetearyl Sulfate) | | | | | | | | 1 | | | |
| Amphisol K Potassium (Cetyl Phosphate) | | | | | | 1 | | | | | |
| Sodium Stearate | | | | | .5 | | | | | | |
| Emulgade PL 68/50 (Cetearyl Glucoside, Cetearyl | | 2.5 | | | | | | | | 4 | |
| Alcohol) | | | | | | | | | | | |
| Tego Care CG (Cetearyl Glucoside) | | | | | | | | | | | 2 |
| Tego Care 450 (Polyglyceryl-3 Methylglucose | | | | | | | | 5 | | | |
| Distearate) | | | | | | | | | | | |
| Cutina MD (Glyceryl Stearate) | | 1 | | 6 | 5 | | 4 | | | 6 | |
| Lanette 14 (Myristyl Alcohol) | | | | 1 | | | | 2 | | | 4 |
| Lanette O (Cetearyl Alcohol) | 4.5 | | 4 | | 1 | 2 | | | | | |
| Novata (Cocoglycerides) | | 1 | | | | | | | | | 1 |
| Lanolin Alcohol | | | | | .5 | .5 | | | | | |
| Lanolin | | | | | | | 5 | | | | |
| Cetiol SB 45 (Butyrospermum Parkii) | | | 1.5 | | | | 2 | | | | |
| Cegesoft C 17 (Myristyl Lactate) | | | | | | | | | | | |
| Myritol PC (Propylene Glycol Dicaprylate/Dicaprate) | | | | | 5 | | | | | | |
| Myritol 331 (Cocoglycerides) | 2 | 5 | 5 | | | 6 | | 12 | | | |
| Finsolv TN (C12/15 Alkyl Benzoate) | | | | | | | | | 8 | | |
| Cetiol CC (Dicaprylyl Carbonate) | 4 | 6 | | | | 4 | 4 | | | | 5 |
| Dicaprylyl Ether (Cetiol OE) | | | | | | | | | 4 | 3 | |
| DC 245 (Cyclomethicone) | | | | | 5 | | | | | | |
| DC 2502 (Cetyl Dimethicone) | | | | | 2 | | | | | | |
| Hydrogenated Polyisobutene | | | | | | | | | | | |
| Dimethicone | .5 | .5 | .5 | | | | 4 | | | | |
| Cetiol 868 (Ethylhexyl Stearate) | | | | | 2 | | 4 | | | | |
| Cetiol J 600 (Oleyl Erucate) | 2 | | 3 | | 3 | 2 | | | | 5 | |
| Ceraphyl 45 (Dioctyl Malate) | | | | | | | 3 | | | | |
| Mineral Oil | | | | 9 | | | | | | | |
| Cetiol SN (Cetearyl Isononanoate) | | | 5 | | | | | | | | |
| Cetiol B (Butyl Adipate) | | | | | | | | | | 2 | |
| Eutanol G (Octyldodecanol) | | | | | | | | | | | |
| Cetiol PGL (Hexyldecanol, Hexyldecyl Laurate) | | | | | | | | | 5 | 5 | |
| Dry Flo (Plus Aluminium Starch Octenyylsuccinate) | 5 | | | | | | | | | | |
| (SFE 839 (Cyclopentasiloxane, | 5 | | | | | | | | | | 2 |
| Dimethicone/Vinyldimethicone Crosspolymer) | | | | | | | | | | | |
| Almond Oil | | | | | | | 1 | | | | |
| Photonyl LS | 2 | 2 | | | | 2 | | | | | |
| Panthenol | 1 | | | | | | | | | | |
| Bisabolol | 0.2 | | | | | | | | | | |
| Tocopherol/Tocopheryl Acetate | 1 | | | | | | | | | | |
| Magnesium Aluminium Silicates (e.g. Veegum types) | | | | | | | | | 1 | | |
| Xantham Gum (e.g. Keltrol types) | | | .4 | | | | | | .5 | | |
| Carbomer (z.B. Carbopol and/or Pemulen types) | .3 | .3 | .3 | .2 | .2 | .2 | | .5 | .1 | .3 | .2 |
| Ethanol | | | | | | | | | | 10 | |
| Butylene Glycol | | | | 4 | 3 | | 2 | 5 | 2 | | |
| Glycerin | 2 | 5 | 5 | | 3 | 3 | 2 | | 4 | | 3 |

| Water, preservative, NaOH (pH 6.5-7.5) | to 100 | | | | | | | | | | |
|---|---|---|---|---|---|---|---|---|---|---|---|
| Component (INCI) | 12 | 13 | 14 | 15 | 16 | 17 | 18 | 19 | 20 | 21 | 22 |
| L = Lotion, C = Cream | C | C | L | C | L | C | L | L | L | L | C |
| Sodium/Potassium 12-Hydroxysterate | 2 | 3 | 1 | 2 | 1 | 2 | 1 | 1 | 1 | 1 | 3 |
| 12-Hydroxystearic Acid | | | 1 | | | | | 1 | | | 1 |
| Eumulgin VL 75 (Polyglyceryl-2 Dipolyhydroxystearate, | | 3 | | | | | 1 | | | | 2 |
| Lauryl Glucoside, Glycerin) | | | | | | | | | | | |
| Dehymuls PGPH (Polyglyceryl-2 | 1 | | | | | | | | | | |
| Dipolyhydroxystearate) | | | | | | | | | | | |
| Generol R (Rapeseed Sterols) | | | | | | 2 | | | | | |
| Eumulgin B2 (Ceteareth-20 | | | | | | 2 | | | | 1 | |
| Polysorbate 60 | | | | | | | | | | 1 | |
| Cutina E 24 (PEG-20 Glyceryl Stearate) | | | | 2 | | | | | | | |
| Hostaphat KL 340 N (Trilaureth-4 Phosphate) | | | | | | | | | | | |
| Lanette E (Sodium Cetearyl Sulfate) | | | | | | | | | | | |
| Amphisol K Potassium (Cetyl Phosphate) | | | | | | | | 1 | | | |
| Sodium Stearate | | | | | 1 | | | | | | |
| Emulgade PL 68/50 (Cetearyl Glucoside, Cetearyl | | 6 | | | | | | 5 | | | 4 |
| Alcohol) | | | | | | | | | | | |
| Tego Care CG (Cetearyl Glucoside) | | | | | | | | | | | |
| Tego Care 450 (Polyglyceryl-3 Methylglucose | | | | | | | | | 4 | | |
| Distearate) | | | | | | | | | | | |
| Cutina MD (Glyceryl Stearate) | 3 | | 3 | 8 | 6 | 8 | | | | 4 | |
| Lanette 14 (Myristyl Alcohol) | | 2 | | | | | | 2 | | 1 | |
| Lanette O (Cetearyl Alcohol) | 2 | | | 2 | | 3 | 1 | | 1 | 1 | 6 |
| Novata (Cocoglycerides) | | | | | | | | | | | |
| Lanolin Alcohol | | | | | | | | | | | |
| Lanolin | | | | | | 4 | | | | | |
| Cetiol SB 45 (Butyrospermum Parkii) | | | | | | | 2 | | | | |
| Cegesoft C 17 (Myristyl Lactate) | 4 | | | | | | | | | | |
| Myritol PC (Propylene Glycol Dicaprylate/Dicaprate) | 6 | | | | | 5 | | | 5 | | |
| Myritol 331 (Cocoglycerides) | 5 | | 5 | | | | 7 | | | 10 | 8 |
| Finsolv TN (C12/15 Alkyl Benzoate) | | 5 | | | 5 | | | 3 | 3 | | |
| Cetiol CC (Dicaprylyl Carbonate) | | | | | | | | | | | |
| Dicaprylyl Ether (Cetiol OE) | | | | | 2 | | 2 | | 5 | | |
| DC 245 (Cyclomethicone) | | 2 | | | 1 | | | | | 8 | |
| DC 2502 (Cetyl Dimethicone) | | 1 | | | 1 | | | | | | 3 |
| Hydrogenated Polyisobutene | 3 | | | | | | | | | | 2 |
| Dimethicone | | | | | 1 | | | | | | |
| Cetiol 868 (Ethylhexyl Stearate) | | 2 | | | | | | | | | |
| Cetiol J 600 (Oleyl Erucate) | | 2 | | | | | | | | | |
| Ceraphyl 45 (Dioctyl Malate) | | | | | | | 3 | | | | |
| Mineral Oil | | | | 10 | | | | | | | |
| Cetiol SN (Cetearyl Isononanoate) | | | | | | | | | | | |
| Cetiol B (Butyl Adipate) | | | 5 | | | 5 | | 4 | | | 3 |
| Eutanol G (Octyldodecanol) | | 3 | 5 | | 5 | | | | | | |
| Cetiol PGL (Hexyldecanol, Hexyldecyl Laurate) | | | | | | | | 5 | 2 | | |
| Dry Flo (Plus Aluminium Starch Octenyylsuccinate) | | 1 | | | | | | | | | |
| (SFE 839 (Cyclopentasiloxane, | | | | | | | | | | | |
| Dimethicone/Vinyldimethicone Crosspolymer) | | | | | | | | | | | |
| Almond Oil | | | | | | 2 | | | | | |
| Photonyl LS | | | | | | 2 | | | | | |
| Panthenol | 1 | | | | | | | | | | |
| Bisabolol | 0.2 | | | | | | | | | | |
| Tocopherol/Tocopheryl Acetate | 1 | | | | | | | | | | |
| Magnesium Aluminium Silicates (e.g. Veegum types) | | | | | | | | | 1 | | |
| Xantham Gum (e.g. Keltrol types) | | | .2 | | | | | | .5 | | |
| Carbomer (z.B. Carbopol and/or Pemulen types) | | .3 | .2 | .3 | | .5 | .2 | .2 | | | .5 |
| Ethanol | | 5 | | 8 | | | | | | | 10 |
| Butylene Glycol | 5 | | 2 | 3 | 3 | | | | | 8 | |
| Glycerin | 2 | 4 | 3 | 3 | | 7 | 5 | 3 | 5 | | |

| | |
|---|---|
| Water, preservative, NaOH (pH 6.5-7.5) | to 100 |

1. Cosmetic and/or pharmaceutical emulsions containing long-chain hydroxyfatty acids and/or salts thereof.

2. Emulsions as claimed in claim 1, characterized in that they contain alkali metal, alkaline earth metal, ammonium, alkyl ammonium, alkanolammonium, glucammonium, aluminium and/or zinc salts of the long-chain hydroxyfatty acids.

3. Emulsions as claimed in claims 1 and/or 2, characterized in that they contain the long-chain hydroxyfatty acids and/or salts thereof in quantities of 0.1 to 10% by weight, based on the final preparation.

4. Emulsions as claimed in at least one of claims 1 to 3, characterized in that they contain 12-hydroxystearic acid.

5. Use of long-chain hydroxyfatty acids and/or salts thereof for the production of waterproof sun protection preparations.

6. Use claimed in claim 1, characterized in that the long-chain hydroxyfatty acids and/or salts thereof are used in quantities of 0.1 to 10% by weight.

7. Use of long-chain hydroxyfatty acids and/or salts thereof as viscosity adjusters.
